(19) United States
(12) Patent Application Publication
(10) Pub. No.: US 2013/0102081 A1
(43) Pub. Date: Apr. 25, 2013

(54) Detection and Characterization of Protein Degradation, Protein Degradation Modulation and Protein Degradation Modulators

(71) Applicants: Ray Clifford Perkins, (US); Sarah-Ellen Perkins Leonard, (US); Paul Johann Andre Kenis, (US)

(72) Inventors: Ray Clifford Perkins, (US); Sarah-Ellen Perkins Leonard, (US); Paul Johann Andre Kenis, (US)

(21) Appl. No.: 13/277,711

(22) Filed: Oct. 20, 2011

(51) Int. Cl. G01N 33/68; G01N 21/64; G01N 27/72

(52) U.S. Cl. 436/86

(57) ABSTRACT

Protein degradation, protein degradation modulation and protein degradation modulators can be detected and characterized through assessment of differential angular mobility exhibited by protein degradation reactants and products.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

Protein degradation (or “degradation”), defined herein as the cleavage of proteins into smaller molecules, occurs via multiple and sometimes overlapping mechanisms. These mechanisms may include radiation exposure, thermal processes, chemical reactions, and enzyme activity. Radiation exposure degradation may arise from but not necessarily be limited to exposure from ambient radiation sources (e.g. sunlight or background radiation), or high-energy sources encountered accidentally or intentionally as in the treatment of disease or irradiation of food, devices and other manufactured goods. Thermal degradation occurs in food production and preparation, burn injuries, and fever associated with disease. Chemical reactions are any and all degradation reactions that do not necessarily involve catalysis by enzymes. Enzyme degradation of proteins includes but is not necessarily limited to hydrolase and lyase activity (Enzyme Commission classes E3 and E4 respectively), a well known exemplar being protease/peptidase activity (Enzyme Commission class E3.4). Thus protein degradation is fundamental to industrial and agricultural processes, the expression and/or treatment of injury and disease, and the functioning of all living organisms. No general methodology exists to detect and characterize protein degradation.

Protein degradation modulation (or “modulation”) as defined herein encompasses any change in degradation including, for example, degradation promotion or degradation inhibition. Protein degradation modulation may arise from changes in physical conditions such as temperature, chemical conditions such as pH and ionic strength, or interactions with chemical and biochemical entities, including other proteins, polynucleotides, cells and cell membranes. For purposes of this invention and due to their special consideration in industrial processes and medicine, the latter category, chemical, biochemical and biological entities, are defined herein as “degradation modulators” (or “modulators”). Certain exemplars are obvious as in the case of class E3.4 enzyme inhibitors and include both naturally occurring and synthetic inhibitors, the latter being a viable objective in drug development and treatment. No general methodology exists to detect or characterize protein degradation modulation or modulators.

A wide range of non-general methods is employed in the detection and characterization of degradation, modulation and modulators. Some are unique to the system under investigation and do not readily apply to other systems or compare to alternate methods. Of the more widely employed methods, the use of peptides as protein surrogates and chromatography, sometimes coupled to mass spectrometry, dominate. The use of peptides, frequently modified to include one or more fluorescent moieties, as protein degradation surrogates has been practiced for decades in the characterization of E3.4 enzymes. Even for this restricted mechanism of degradation, the use of peptides as protein degradation surrogates is problematic. Cleavage of peptides does not predict the kind or number of whole-protein substrates for a given enzyme, or whole-protein targets of broader degradation mechanisms. A peptide cannot substitute as a protein in complex, biologically relevant systems rendering the method unsuitable for modulator determinations. For example, peptide interactions with non-degradation reactants are typically undetectable. Chromatography, whether or not coupled to mass spectrometry, by its very nature disturbs equilibria as components separate. Chromatography as applied to degradation is not amenable to continuous reaction observation demanding, instead, sampling at discrete intervals. More problematic is the inability to incorporate bio-relevant entities such as cells or cell fragments as potential modulators of degradation.

Regardless of degradation mechanism, degradation always results in products with lower molecular weight than reactants and, therefore, products exhibit greater mobility, both translational and angular. Angular mobility is more strongly dependent on changes in molecular weight and, thus, the observable of choice. Of extant methods sensitive to changes in angular mobility, electron magnetic resonance (EMR, also known as Electron Spin Resonance (ESR) or Electron Paramagnetic Resonance (EPR)) and fluorescence polarization (FP) spectroscopies dominate. Other observables may change upon degradation detectable by the cited spectroscopies. Exemplar is EMR detection of changes in site polarity and, therefore, site polarity changes may serve as an observable for degradation, modulation and modulators.

### SUMMARY OF THE INVENTION

This invention demonstrates the utility of angular mobility measurements as a general-purpose approach in the detection and characterization of protein degradation (the cleavage of proteins into smaller molecules), protein degradation modulation and protein degradation modulators. At the core of the approach, the degradation of whole proteins may be continuously monitored in real time with changes in angular mobility differentiating products from reactants. Degradation modulation is readily observed in comparative measurements and the identity and behavior of degradation modulators verified and assessed. This invention accommodates observations in systems of any desired biological complexity including but not limited to the addition of other proteins, polynucleotides, membrane fragments and whole cells. Thus, protein degradation, degradation modulation and degradation modulators may be assessed in bio-relevant media of any desired complexity. This is crucial to prediction of degradation modulation and modulator behavior in vivo.

### BRIEF DESCRIPTION OF THE DRAWINGS

Drawing 1 of 3 shows angular mobility differentiation of reactants and products for whole-protein degradation;

Drawing 2 of 3 shows complete inhibition of whole-protein degradation by a known degradation modulator;

Drawing 3 of 3 shows the time course of degradation products with and without variably effective modulators;

### DETAILED DESCRIPTION OF THE INVENTION

This invention exploits changes in angular mobility that must accompany protein degradation (“degradation”), the cleavage of proteins into smaller molecules, regardless of degradation mechanism. The invention utilizes measurement methods sensitive to angular mobility to detect and characterize degradation. The ability to characterize degradation per se encompasses, through direct comparison of data, protein degradation modulation (“modulation”) regardless of the modulation mechanism. Chemical, biochemical or biological entities that act as protein degradation modulators (“modulators”) are readily identified and their actions characterized. Modulator interactions with degradation participants or any other chemical, biochemical or biological entity may also be assessed. Multiple embodiments of the invention include but are not limited to Electron Magnetic Resonance (EMR, also known as Electron Spin Resonance (ESR) or Electron Paramagnetic Resonance (EPR)) spectroscopy in all modes of operation in combination with spin labeling or performed on native paramagnetic centers, Fluorescence Polarization (FP) spectroscopy in combination with fluorescent labeling or performed on native fluorescent centers, and Nuclear Magnetic Resonance (NMR) spectroscopy in all modes of operation in combination with isotopic substitution or performed on native nuclei. In the case of EMR and NMR, degradation may be expressed through angular mobility changes and/or polarity changes at the paramagnetic or spin loci.

This invention uniquely accommodates continuous, real time observation of whole-protein degradation in media of any desired complexity. Any and all proteins degraded by any and all mechanisms are detectable, and the degradation process is subject to characterization. Likewise, modulation driven by modulator interaction with the degradable protein is detectable and the interaction subject to characterization. This invention requires no separation of reaction media so that all crucial equilibria are maintained, in contrast to chromatography. Finally, this invention allows observation of degradation and attendant modulation in the presence of, for example cells and cell membranes, a benefit of significant value but not realized via other methods.

One embodiment of the invention, data shown herein, demonstrates the utility of EMR spectroscopy in combination with spin labeling. The spin label is attached to a protein and the control or un-degraded protein EMR spectrum recorded. Degradation of the protein by any degradation mechanism creates products with predictably different angular mobility than that of the control. Data included herein demonstrate that the increase in angular mobility for degradation products relative to the un-degraded reactant is expressed as an identifiable subpopulation in the EMR spectrum. Therefore degradation per se is demonstrated through observation that the EMR lineshape is altered. Degradation is further characterized through quantitative analysis of the product and reactant subpopulations. Modulation of that degradation, regardless of the mechanism of modulation, can be expressed in a variety of ways, only one of which is an alteration in concentration of the degradation subpopulations. For example, inhibition of degradation is expressed by a reduction in concentration of relatively mobile subpopulation(s), degradation promotion by an increase in the mobile subpopulation. Chemical, biochemical and biological entities are readily tested for their potential as modulators.

Drawing 1 of 5 demonstrates the detection and characterization of protein degradation. Shown are EMR spectra of spin-labeled bovine serum albumin (BSA, spin label attached to BSA under conditions which favor attachment to a sulfhydryl). Key features of the intact protein's spectrum are denoted by the numbers 1 and 5. At 7, 21 and 40 minutes (spectra 2, 3 and 4 respectively) following the addition of the protease, trypsin, a second, more mobile, product population appears, characterized by three, relatively sharp peaks superimposed on the spectrum of the intact protein. Protein degradation is clearly expressed with products expressing more rapid angular mobility than reactants. Both reactant and product subpopulations are apparent in the spectra with the growth of the product population matched by a concomitant loss of reactants. Subpopulation spectra are amenable to further analyses in accord with practices associated with the selected observation method.

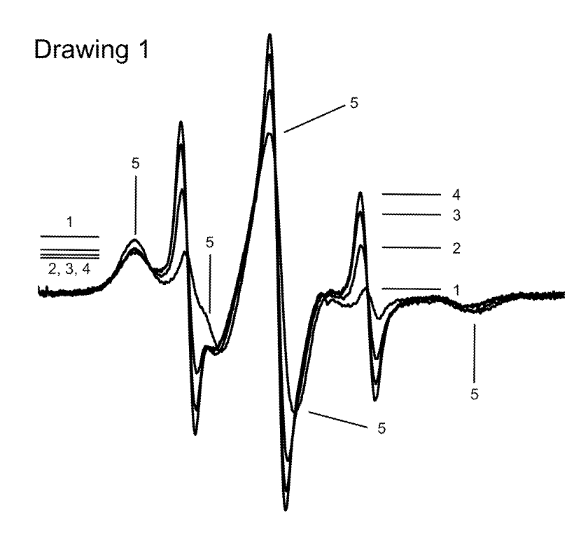

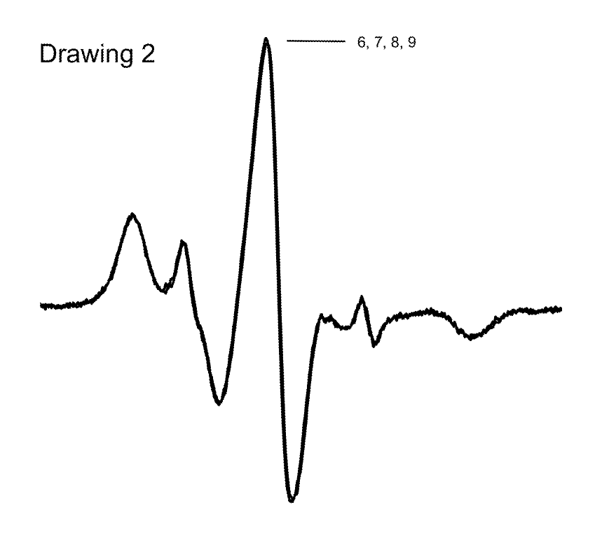

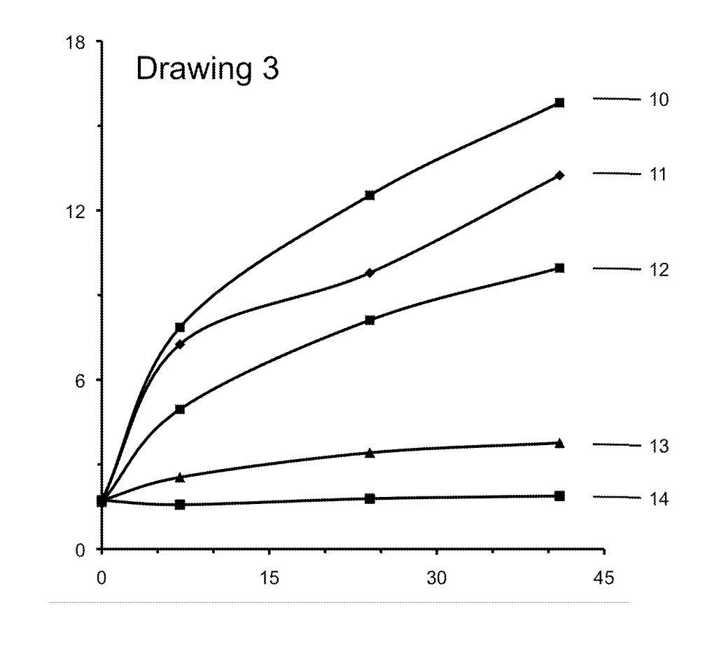

Drawing 2 of 5 demonstrates the detection and characterization of a protein degradation modulator. Shown are four EMR spectra (6, 7, 8, 9) for conditions identical to those in FIG. 1 of 5 with the exception that trypsin inhibitor is present. The presence and action of the modulator is clear and unambiguous.

Drawing 3 of 5 demonstrates modulator detection and characterization via subpopulation kinetic analyses. Shown is the concentration of products, as reflected in the heights of the rightmost peaks labeled 1, 2, 3, and 4 in FIG. 1 of 5, versus time. These data reflect the degradation of BSA by the protease, trypsin. The time course of appearance of the mobile subpopulation is shown for protease+the drug Nelfinavir (10), protease alone (11), protease+chymostatin (12), protease+leupeptin hemisulfate (13), and protease+trypsin inhibitor (14). Modulation of protein degradation is clear for each modulator and the direction and degree of modulation for 12, 13 and 14 is in accord with prior results obtained from other methods.

Drawing 3 of 5 also demonstrates detection and characterization of modulator interactions. The near-complete suppression of degradation by trypsin inhibitor (14) is construed as inhibitor interaction with the protease. By contrast promotion of degradation by Nelfinavir (10), in use as a presumed inhibitor of HIV protease, is more consistent with drug interaction with the substrate.

Test materials, whether natural, expressed or synthetic, may be obtained from any and all sources.

The term “Protein” as used herein encompasses all naturally occurring proteins, modified naturally occurring proteins, protein fragments, protein degradation products, expressed and/or synthetic proteins, and polypeptides.

The term “Protein Degradation” is used in the most general sense as an event or events in which a protein is divided into two or more molecules by any process or mechanism.

The term “Protein Degradation Modulation” is used in its most general sense as any change in protein degradation relative to the non-modulated results. Modulation may include but not be limited to qualitative changes in the data or quantitative changes in reaction and equilibrium kinetics, reaction mechanism determination, association-dissociation constants, and stoichiometric dependences.

The term “Protein Degradation Modulator” is used in its most general sense as any entity, change in test materials, or change in test conditions that effects a change in protein degradation. Modulators may include but not be limited to changes in temperature, acidity (basicity), or the introduction of one or more chemical, biochemical or biological entities. Exemplar of a “Modulator” is any chemical, biochemical or biological entity in use or intended for use as a pharmaceutical agent.

“Angular mobility” is construed as the angular displacement in time of the observed protein and/or protein fragments as solid-body equivalents combined with local, angular displacement events. In other contexts “angular mobility” is the composite of rotational, segmental and local angular displacement phenomena.

The criterion for selection of detection method is, in part, dependence of the detection method on the mobility of the observed site(s). EMR in any and all its modes of operation combined with spin labeling is one such detection method. Others may include but not be limited to Fluorescence Polarization and fluorescent labeling, and Nuclear Magnetic Resonance spectroscopy performed in any and all of its modes of operation, with or without isotopic substitution.

Data generated via these methods encompasses the qualitative observation of protein degradation and degradation modulation per se, and the quantitative determination of, but not limited to, reaction and equilibrium kinetics, reaction mechanism determination, association/dissociation constants, and stoichiometric dependences.

In broad embodiment, the present invention is a set of related methods for detection and characterization of protein degradation, protein degradation modulation and protein degradation modulators via assessment of differential angular mobility of protein degradation reactants and products.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

We claim:

1. Detection and characterization of protein degradation, the cleavage of proteins into smaller molecules, regardless of degradation mechanism(s) via detection of changes in angular mobility of proteins consistent with their degradation.
1.1. claim No. 1 performed with any method or methods capable of detection of changes in angular mobility including but not limited to Electron Magnetic Resonance spectroscopy (EMR, also known as Electron Spin and Electron Paramagnetic Resonance spectroscopies) in all modes of operation, various Fluorescent spectroscopies and Nuclear Magnetic Resonance (NMR) spectroscopy in all modes of operation.
1.2. claim No. 1 performed via EMR spectroscopy in all modes of operation either combined with Spin Labeling and/or through observation of native EMR-responsive moieties.
1.3. claim No. 1 performed via Fluorescence Polarization either combined with Fluorescent Labeling and/or through observation of native fluorescent moieties.
1.4. claim No. 1 performed via Nuclear Magnetic Resonance (NMR) spectroscopy in all modes of operation either combined with isotopic substitution and/or observation of native NMR-responsive nuclei.
1.5. claim No. 1 performed with EMR or NMR spectroscopies in all modes of their operation for degradation detection afforded in whole or in part by degradation-induced changes in polarity of the paramagnetic or spin loci.

2. Detection and characterization of protein degradation modulation including but not limited to degradation promotion and inhibition via detection of changes in angular mobility of proteins consistent with their degradation.
2.1. claim No. 2 performed with any method or methods capable of detection of changes in angular mobility including but not limited to Electron Magnetic Resonance spectroscopy in all modes of operation (EMR, also known as Electron Spin and Electron Paramagnetic Resonance spectroscopies), various Fluorescent spectroscopies and Nuclear Magnetic Resonance (NMR) spectroscopy.
2.2. claim No. 2 performed via EMR spectroscopy in all modes of operation either combined with Spin Labeling and/or through observation of native EMR-responsive moieties.
2.3. claim No. 2 performed via Fluorescence Polarization either combined with Fluorescent Labeling and/or through observation of native fluorescent moieties.
2.4. claim No. 2 performed via NMR spectroscopy in all modes of operation either combined with isotopic substitution and/or observation of NMR-responsive nuclei.
2.5. claim No. 2 performed with EMR or NMR spectroscopies in all modes of their operation for degradation detection afforded in whole or in part by degradation-induced changes in polarity of the paramagnetic or spin loci.

3. Detection and characterization of protein degradation modulators including but not limited to degradation promoters and inhibitors via detection of changes in angular mobility of proteins consistent with their degradation.
3.1. claim No. 3 performed with any method or methods capable of detection of changes in angular mobility including but not limited to Electron Magnetic Resonance spectroscopy in all modes of operation (EMR, also known as Electron Spin and Electron Paramagnetic Resonance spectroscopies), various Fluorescent spectroscopies and Nuclear Magnetic Resonance (NMR) spectroscopy.
3.2. claim No. 3 performed via EMR spectroscopy in all modes of operation either combined with Spin Labeling and/or through observation of native EMR-responsive moieties.
3.3. claim No. 3 performed via Fluorescence Polarization either combined with Fluorescent Labeling and/or through observation of native fluorescent moieties.
3.4. claim No. 3 performed via NMR spectroscopy in all modes of operation either combined with isotopic substitution and/or observation of NMR-responsive nuclei.
3.5. claim No. 3 performed with EMR or NMR spectroscopies in all modes of their operation for degradation detection afforded in whole or in part by degradation-induced changes in polarity of the paramagnetic or spin loci.
